(19) United States
(12) Patent Application Publication
(10) Pub. No.: US 2014/0142177 A1
(43) Pub. Date: May 22, 2014

(54) TOPICAL ORGANIC ACID SALT COMPOSITIONS SUITABLE FOR TREATING INFECTIONS

(71) Applicants: Peter Mladenovich, Oakville (CA); William D. Lougheed, Toronto (CA)

(72) Inventors: Peter Mladenovich, Oakville (CA); William D. Lougheed, Toronto (CA)

(21) Appl. No.: 13/884,927

(22) PCT Filed: Nov. 12, 2010
(86) PCT No.: PCT/CA2010/001812

§ 371 Date: Jan. 16, 2014

Related U.S. Application Data
(63) Continuation-in-part of application No. 12/466,615, filed on May 15, 2009.

(51) Int. Cl. A61K 31/315; A61K 9/00; A61K 47/10; A61K 31/19; A61K 31/192

(52) U.S. Cl. CPC A61K 31/315; A61K 31/19; A61K 31/192; A61K 47/10; A61K 9/0014
USPC 514/494

(57) ABSTRACT

A non-toxic, topical antifungal composition and use of the composition for treating infections comprising a combination of at least three or more low molecular weight, hydrophilic organic acid salts and/or derivatives exhibiting a synergistic enhancement, dissolved in a carrier, wherein the low molecular weight organic acid salts are selected from the group consisting of saturated and unsaturated, acyclic, branched and unbranched aliphatic carboxylic acids wherein the longest carbon chain has eight carbons, and aromatic carboxylic acids containing less than ten carbon atoms; the carrier comprises one or more non-aqueous, non-volatile polyhydric solvents; and wherein the combination of low molecular weight, hydrophilic organic acid salts or derivatives comprises between about 0.5% to about 50 by weight of the composition, and no single acid salt or derivative comprises more than about 75% by weight of the total acid content.

(This application is a continuation-in-part of U.S. application Ser. No. 12/466,615 filed May 15, 2009 whose contents are incorporated herein by reference.)

### FIELD OF THE INVENTION

The present invention relates to non-aqueous topical compositions of low molecular weight, hydrophilic carboxylic acid salts and/or derivatives for treating toe and finger nail fungal infections (onychomycosis), as well as fungal infections of the skin. Tinea pedis (athlete's foot), Tinea unguium (nail infections), Tinea cruris (jack itch), Tinea corporis, Tinea versicolor and Tinea candidiasis, among others, are examples of onychomycosis and fungal infection. Each are caused by different types of fungus such as those of the gena Trichophyton, Epidermophyton, Microsporum and Candida. The present invention also relates to the use of the non-aqueous topical compositions for treating herpes infections.

### BACKGROUND OF THE INVENTION

Fungal infection of the nails is one of the most common diseases of the nail bed or plate. It is estimated that between 6 and 8 percent of the adult population is affected by such fungal infections to a varying degree. Fungal infections of the nail (onychomycosis) are often caused by dermatophytic fungi, most often by one of Trichophyton mentagrophytes (also known as Trichophyton interdigitale) and Trichophyton rubrum, although numerous other fungi are also known to cause such infections. Fungal infection of the skin such as Tinea pedis (athlete's foot), Tinea cruris (jock itch) and Tinea corporis are also commonly caused by T. rubrum and T. mentagrophytes, among other fungi. T. mentagrophytes is generally easily treated by conventional over-the-counter (OTC) medications whereas T. rubrum is significantly more difficult to treat.

Herpes is an infection that is caused by a herpes simplex virus. Oral herpes causes cold sores around the mouth and face. Genital herpes affects the genitals, buttocks or anal area. Herpes has no cure and can only be controlled by powerful anti-viral drugs.

A large number of pharmaceutical compositions for use in the topical treatment of skin and nail fungal infections have been described in the art. These topical applications include lotions, sprays, gels, ointments and powders containing a variety of prescription and non-prescription active ingredients.

U.S. Pat. No. 6,080,744 describes cream-based topical treatments for mycotic infections consisting of a blend containing multiple active ingredients including ketaconazole, nystatin, miconazole nitrate, tolnaftate, clotrimazole, undecenoic acid, zinc undecenoate, propionic acid and sodium propionate. Optionally, the compositions may include additional active ingredients such as an antibacterial agent (e.g., gentamicin) or an anti-inflammatory agent (e.g., dipropionate betamethasone). However, the testing data only demonstrates activity against skin infections and there is no discussion of infections of the nail.

U.S. Pat. No. 6,664,292 describes compositions for the treatment of fungal infections of the nail comprised of a lower alcohol, preferably methanol, and a single, lower carboxylic acid. The composition is extremely volatile, however, and requires special storage considerations in order to prevent the methanol from evaporating.

U.S. Pat. No. 4,824,865 describes compositions containing 2-hydroxyoctanoic acid, 2-ketooctanoic acid and O₂ to C₆ esters thereof in aqueous and non-aqueous formulations for the treatment of various skin infections. There is no discussion of nail infections and the acidic pH of the water-containing formulations would result in irritation to the user.

U.S. Pat. No. 6,214,889 describes liquid and gel compositions for the treatment of adverse skin conditions consisting of one or more of potassium, sodium, calcium or cesium formate alone or in combination, preferably in concentrations of about 50% in distilled water and an optional gelling agent. The composition of this invention contains a very large amount of water and there is no teaching that it would be effective against nail infections.

U.S. Pat. No. 6,921,529 describes a topical composition containing a weak organic acid dispersed in a non-biodegradeable polymeric matrix to form a supersaturated hydrogel. The polymer is preferably a polyacrylate and the weak organic acid is acetic or citric acid. Optionally, the use of other, known, antimycotic agents such as azole derivatives, undecylenic acid, tea tree oil (Melaleuca alternifolia), or salicyclic acid may be added to the hydrogel. By definition a hydrogel contains a large amount of water and the use of these acids would result in an irritation to the end user

Nettleship (Archives of Dermatology and Syphilology, 1950, 61(4), 669) discusses the use of a propionic acid-propionate-caprylate mixture in ointment or powder form to treat skin infections. Application of the ointment containing 12.3% sodium propionate, 2.7% propionic acid, 10% sodium caprylate and 5% zinc caprylate and subsequent application of the powder containing 15% calcium propionate, 5% zinc propionate, 5% zinc caprylate and 0.25% propionic acid were required to effect treatment. In addition, there is no discussion of treatment of nail infections.

Brewer (Archives of Dermatology and Syphilology, 1950, 61(4), 681) discusses the use of “sopronol” (a mixture of 12.3% sodium propionate, 2.7% propionic acid and 10% sodium caprylate) in powder or water-soluble ointment to treat mild to severe skin infections. Ointment application to the skin and powder application to socks and footwear was required. Moreover, there is no discussion of treatment of nail infections.

Sulzberger and Kanof (Archives of Dermatology and Syphilology, 1947, 55(3), 391) discuss the use of “sopronol” (a mixture of 5% zinc propionate and 15% calcium propionate) powder in comparison with other compositions for the prevention and treatment of skin infections of the feet. There is no discussion of the use of these compositions for treating nail infections.

Peck and Russ (Archives of Dermatology and Syphilology, 1947, 56(5), 601) discuss the use of an aqueous ointment containing 12.3% sodium propionate, 2.7% propionic acid and 10% sodium caprylate to treat tinea infections of the scalp, rectum, nails and hands and feet. Treatment of the nails was cumbersome and arduous, requiring grinding of the nails until pain was induced, application of the ointment and alternating filing of the nails and application of the ointment twice a day for several months.

Mori et al (Agricultural and Biological Chemistry, 1987, 51(12) 3403) examined the activity of various saturated and unsaturated fatty acids against Pyricularia oryzae and Miscanthus sinensis and concluded C₁₀ to C₁₃ monocarboxylic fatty acids were the most active. However, there is no discussion of clinical treatment of skin and nail infections.

Kabara et al (Antimicrobial Agents and Chemotherapy, 1972, 2(1), 23) evaluated the activity in vitro of 15 saturated and unsaturated fatty acids against gram positive and gram negative bacteria. Lauric (C₁₂) acid was found to be the most effective. However, there was no discussion of clinical treatment of skin and nail infections.

Garg and Muller (Mycoses, 1993, 36, 51) examined the in vitro antifungal activity of saturated and unsaturated C₇ to C₁₈ fatty acids against Trichophyton and Microsporum species that cause fungal infection. C₇ to C₁₁ saturated fatty acids were most active against fungi that cause skin infection. However, there was no discussion of clinical treatment of skin and nail infection.

While these teachings describe topical compositions for the treatment of fungal infections, most of these compositions have a high loading of active ingredient, cause undesirable side effects and require an invasive or long treatment regimen.

Further, fungal infections of nails (onychomycosis) are very difficult to treat with topical formulations because the fungus is often embedded deep within the nail and nail bed. This is due to the physiological make up of the nail which precludes proper absorption of the antifungal treatment. These factors can contribute to a treatment regimen which offers no guarantee of fungus eradication because of ineffectiveness or noncompliance with the administration guidelines.

As a result, there remains a long-felt need for new, safe and inexpensive compositions for the effective treatment of fungal infections of both skin and nails, and viral infections of the skin.

### SUMMARY OF THE INVENTION

The present invention relates to a non-toxic, topical antifungal composition for the treatment of fungal infections of the nails and skin. The active ingredients of the antifungal composition comprise a combination of low molecular weight, hydrophilic carboxylic acid salts or derivatives exhibiting a synergistic enhancement, dissolved in a non-aqueous, non-volatile polyhydric carrier. The composition can also contain a nail-penetrating agent to allow absorption of the composition into the nail. Since the source of onychomycosis can be traced to the nail bed, allowing the low molecular weight, hydrophilic carboxylic acid salts and/or derivatives to penetrate to the nail bed results in the most effective treatment of the fungal infection.

While the low molecular weight, hydrophilic carboxylic acids of the composition individually have each been previously known to demonstrate weak antifungal activity, it unexpectedly has been found that when they are used in combination, a synergistic enhancement and a potent effect is observed. Synergistic is defined as providing more than an additive effect of the combination. This still greater enhancement has allowed for a non-toxic, topical antifungal formulation with a lower amount of low molecular weight, hydrophilic carboxylic acid salts or derivatives than had been used in the prior art.

Thus, in one aspect of the invention there is provided a composition suitable for the topical treatment of a variety of fungal infections that may develop on the skin and nails comprising a combination of low molecular weight, hydrophilic organic acid salts or derivatives exhibiting a synergistic enhancement, dissolved in a non-aqueous, non-volatile polyhydric carrier.

In another aspect of the invention there is provided a composition comprising a combination of low molecular weight, hydrophilic organic acid salts or derivatives exhibiting a synergistic enhancement and a penetrating agent, dissolved in a non-aqueous, non-volatile polyhydric carrier used to treat onychomycosis.

In another aspect of the invention there is provided a composition comprising a combination of low molecular weight organic acid salts or derivatives exhibiting a synergistic enhancement and a penetrating agent, dissolved in a non-aqueous, non-volatile polyhydric carrier used to treat fungal infection.

In a further aspect of the invention there is provided a method of treatment using a composition comprising a combination of low molecular weight, hydrophilic carboxylic acid salts or derivatives exhibiting a synergistic enhancement and a penetrating agent, dissolved in a non-aqueous, non-volatile polyhydric solvent.

In a further aspect of the invention there is provided a combination of low molecular weight, hydrophilic carboxylic acid salts or derivatives exhibiting a synergistic enhancement useful in treating onychomycosis and fungal infection.

In a further aspect of the invention there is provided a method of treating skin and nails using a combination of low molecular weight, hydrophilic carboxylic acid salts or derivatives exhibiting a synergistic enhancement at a lower concentration than previously used.

In a further aspect of the invention there is provided a composition having an increased drug flux comprising a supersaturated concentration of low molecular weight, hydrophilic organic acid salts or derivatives.

According to another aspect of the invention we have discovered unexpectedly that the invention is suitable for use for the treatment of genital herpes and particularly herpes cold sores

### MORE DETAILED DESCRIPTION OF ASPECTS OF THE INVENTION

According to another aspect of the invention there is provided a non-toxic, topical antifungal composition comprising a combination of at least three or more low molecular weight, hydrophilic organic acid salts or derivatives exhibiting a still greater synergistic enhancement, dissolved in a carrier, wherein
the low molecular weight organic acid salts are selected from the group consisting of saturated and unsaturated, acyclic, branched and unbranched aliphatic carboxylic acids wherein the longest carbon chain has eight carbons, and aromatic carboxylic acids containing less than ten carbon atoms;
the carrier is comprised of one or more non-aqueous, non-volatile polyhydric solvents; and
wherein the combination of low molecular weight, hydrophilic organic acid salts and/or derivatives comprises between about 0.5% to about 50%, preferably to about 30% and most preferably to about 10% by weight of the composition, and no single acid salt or derivative comprises more than about 75% by weight of the total acid content.

According to another aspect of the invention the active ingredients of the present antifungal compositions are combinations of at least three or more low molecular weight, hydrophilic organic acid salts or derivatives. These low molecular weight, hydrophilic organic acids include both substituted and non-substituted aliphatic (saturated and unsaturated) and aromatic acids. Preferably, the carboxylic acid has less than 12, and more preferably less than ten carbons, with the longest carbon chain being eight carbon atoms in length and the aromatic carboxylic acid containing less than ten carbon atoms. The carboxylic acids used in the present invention can be substituted by one or more functional groups, such as alkyl, alkenyl, alkynyl, halogen, hydroxy, carbonyl, carboxylic acid, aldehyde, ester, amide, carbonate, carbamate, ether, amino, cyano, isocyano, oxy, oxo, thia, aza, azide, imine, nitro, nitrate, nitroso, nitrosooxy, cyanate, isocyanate, thiocyanate, isothiocyanate, sulfinyl, sulfhydryl, sulfonyl, phosphino. Each of the alkyl, alkenyl, alkynyl and amino groups may themselves be optionally substituted with one or more of the preceding functional groups.

Preferably, the carboxylic acids are selected from branched and unbranched alkanoic acids, hydroxyalkanoic acids, alkenoic acid, aromatic acids, and hydroxyaromatic acids. Such organic acids include formic, acetic, propionic, butyric, valeric, caproic, enanthic, caprylic, lactic, tartaric, gluconic, benzoic, mandelic, salicylic, acrylic, acetoacetic, pyruvic, adipic, aldaric, fumaric, glutaric, maleic, sorbic, malic, malonic, oxalic, succinic, tartronic, citric, isocitric, aconitic, and carballylic acids, as well as their further branched and substituted derivatives.

The salts of the carboxylic acids most useful in the present invention are preferably metal salts. More preferably, the metal salts are selected from the usual groups 1 and 2 metals and zinc. Groups 1 and 2 metal salts normally comprise lithium, sodium potassium, magnesium and calcium salts. Most preferably the metal salts used in the present invention are sodium, calcium and zinc.

Most preferred active ingredients for the composition of the present invention are sodium acetate, sodium formate, zinc propionate, calcium propionate and sodium benzoate, and various combinations thereof. These compounds and other salts and/or derivatives can be grouped together in various amounts and in various numbers and provides substantially the same benefits, and groups of three, four or five salts and/or derivatives may be chosen to achieve the benefits of the invention.

It unexpectedly has been found that the compositions of the invention are most potent when the carrier system is non-aqueous. Non-aqueous in this case means low to negligible water content, i.e. 5% and more preferably less than about 1% water as formulated based on the weight of the composition. The solvents used for the present compositions will preferably have a low to negligible water content. Thus, the low molecular weight, hydrophilic carboxylic acid salts or derivatives of the present compositions are dissolved in a non-aqueous, non-volatile polyhydric solvent. Polyhydric in this case means containing more than one hydroxyl group. Preferably, the solvents include glycerine, diglycerol, ethylene glycol, propylene glycol, low molecular weight polyglycols, such as polyethylene glycol or polypropylene glycol with molecular weight less than 500 gmol⁻¹, among others, and their derivatives, particularly monoether and ester derivatives. More preferred as solvents are glycerine and propylene glycol. Most preferred as a solvent is propylene glycol.

The compositions of the present invention can also comprise from between about 1% to about 80% preferably between about 30% and about 80%, more preferably between about 50% to about 70% of a penetrating agent to allow the low molecular weight, hydrophilic carboxylic acid salts or derivatives to penetrate the nail itself to ensure the carboxylic acid salts or derivatives contact the source of the fungal infection, usually the nail bed. The penetrating agent may be selected from alcohols, preferably lower alkanols. More preferred alkanols are methanol, ethanol, n-propanol, isopropanol, n-butanol, sec-butanol and tert-butanol. Most preferred are isopropanol and ethanol.

An optional gel-forming component may be incorporated in the compositions of the invention to produce a formulation suitable for application to bandages, dressings and the like. Preferred gel-forming ingredients are hydroxyl ethyl cellulose or a fumed silica (Aerosil®) which may be used in non-aqueous solvents or carriers.

Two preferred compositions of the present invention comprise the combination of sodium acetate, sodium formate, zinc propionate, calcium propionate, and sodium benzoate or the above without sodium acetate in the non-aqueous, non-volatile polyhydric carrier propylene glycol, to which is added the penetrating agent ethanol or isopropanol. Glycerol may optionally be added to alter the viscosity of the compositions of the present invention.

The combination of low molecular weight, hydrophilic carboxylic acid salts and/or derivatives in the present compositions may be added to the carrier at a level between about 1% to about 50% total carboxylic acid salt or derivative content by weight of the composition. Preferably, the total combination of carboxylic acid salts or derivatives is between about 1% to about 30% by weight of the composition, and more preferably the total synergistic combination of carboxylic acid salts or derivatives is between about 1% to about 20% by weight of the composition. The distribution of the individual carboxylic acid salts or derivatives within the total combination of carboxylic acid salts or derivatives is not limited, however. It is preferable that any one carboxylic acid salt or derivative does not comprise more than about 75% of the combination. Preferably, the individual carboxylic acid salts and/or derivatives are added to the carrier at a level of between about 1% to about 10% by weight of the total formulation.

The compositions of the present invention exhibit antifungal properties and are therefore useful in the topical treatment against fungi which cause onychomycosis and fungal infection such as tinea unguium, tinea pedis, tinea cruris, tinea corporis, tinea versicolor and tinea candidiasis. The fungi causing these tinea infections include the various species of Trichophyton, Epidermophyton, Microsporum and Candida. The compositions of the present invention are particularly useful in treating onychomycoses caused by T. mentagrophytes (also known as T. interdigitale) and T. rubrum, in particular T. rubrum.

As well as being applied directly to the infected area, compositions of the invention can also be pre-applied to bandages or other absorbent material, which is then applied to the infected area. Alternatively, the treated area can be wrapped in a bandage or other protective covering.

Further, the use of the compositions described herein as a medicament is provided for the topical treatment of cold sores, in one embodiment herpes cold sores.

Thus, in accordance with the invention, methods of treatment of onychomycosis and fungal infection have been provided wherein an effective amount of the compositions described herein is liberally applied to the infected area by applying by hand or painting. As well, the use of the compositions described herein for the topical treatment of onychomycosis and fungal infection has also been provided. Topical treatment of fungal infection may include, but not be limited to, application of a gel formulation of the compositions of the present invention to the skin with repeated, daily application of the gel formulation until eradication of the fungal infection is achieved, for example singularly or repeatedly. Topical treatment of onychomycosis may include, but not be limited to, painting the infected nail or nails with a liquid formulation of the compositions of the present invention with repeated, daily application of the liquid formulation until eradication of the fungal infection is achieved, for example singularly or repeatedly. Optionally a bandage or dressing impregnated with a composition of the present invention further can be applied to maintain a steady level of the antifungal composition at the treatment site. Other formulations of the compositions of the present invention that may be used to treat fungal infections of the skin and nails may include creams, lotions, ointments and the like.

Further, the use of the compositions described herein in the preparation of a medicament for the topical treatment of onychomycosis and fungal infection is also provided.

Further, the use of the compositions of the invention in the preparation of a medicament for the topical treatment of Candida infections is also provided.

Exemplary Formulations of the Herpes/Antifungal Compositions of the Invention

Formulation of the antifungal compositions of the present invention may be prepared by the methods known to the skilled person. Two exemplary, but non-limiting, formulations of the present invention, a liquid and a gel formulation, are provided below. In addition to these exemplary formulations, other topical formulations, prepared using methods known to the skilled person, may also be used for the administration of the antifungal compositions of the present invention.

### EXAMPLE 1

### Liquid Formulation

A preferred liquid formulation having low to negligible water content contains the following ingredients:

| Ingredient | % by Wt. |
|---|---|
| Propylene glycol | 72 |
| Sodium benzoate | 5 |
| Calcium propionate | 5 |
| Zinc propionate | 5 |
| Sodium formate | 3 |
| Glycerol | 10 |

This formulation may be prepared by, among other methods, first warming the propylene glycol and sequentially dissolving in it, the sodium benzoate, calcium propionate, zinc propionate and sodium formate. After dissolution of each of the salts, the glycerol is added, following which the mixture is cooled to room temperature.

### EXAMPLE 2

### Gel Formulation

A preferred gel formulation having low to negligible water content contains the following ingredients:

| Ingredient | % by Wt. |
|---|---|
| Propylene glycol | 80 |
| Sodium benzoate | 5 |
| Calcium propionate | 5 |
| Zinc propionate | 5 |
| Sodium formate | 3 |
| Hydroxy ethyl cellulose | 2 |

This formulation may be prepared by, among other methods, first warming the propylene glycol and sequentially dissolving in it the sodium benzoate, calcium propionate, zinc propionate and sodium formate. After dissolution of each of the salts, the hydroxy ethyl cellulose is slowly added to prevent agglomeration, following which the mixture is cooled to room temperature.

Use and Efficacy of the Antifungal Compositions of the Invention

### EXAMPLE 3

### Confirmation of the Antifungal Activity of the Antifunqal Compositions—Liquid Broth Assay

The antifungal activity of the present compositions was confirmed through in vitro testing using an antifungal microdilution method using Canadian Laboratory Standards Institute (CLSI) Reference Method 38-A2 with modifications as described below. This method is the gold standard used in measuring the antifungal susceptibility of filamentous fungi that cause invasive infections. Fungal colonies are grown on potato glucose agar (PGA). One colony is picked and grown in Sabouraud glucose broth (SGB) at 24° C. for 3 days in the presence of test compound. Microdilution trays are incubated at 24° C., and are read after 5 days of culture. Turbidity in the microdilution wells is scored with the aid of a reading mirror and compared with that of the growth control. A numerical score from 0 to 4 is given to each well using the following scale: 0=optically clear or absence of growth, 1=slight growth (25% of growth control), 2=prominent reduction in growth (50% of growth control), 3=slight reduction in growth (75% of growth control), 4=no reduction in growth.

The turbidity scores (average of 3 tests) for T. rubrum and T. mentragrophytes grown in different concentrations (or dilutions) of test compounds for 5 days was as follows:

Trichophyton rubrum

| | Purity (%) | | | | | |
|---|---|---|---|---|---|---|
| | 0 | 0.625 | 1.25 | 2.5 | 5 | 10 |
| Formulation of Ex. 1 | 4 | 0 | 0 | 0 | 0 | 0 |
| Carrier | 4 | 4 | 4 | 4 | 2.67 | 1 |

Trichophyton mentagrophytes

| | Purity (%) | | | | | |
|---|---|---|---|---|---|---|
| | 0 | 0.625 | 1.25 | 2.5 | 5 | 10 |
| Formulation of Ex. 1 | 4 | 1 | 1 | 0.67 | 0 | 0 |
| Carrier | 4 | 4 | 4 | 4 | 3 | 2 |

The compositions of the current invention contain several carboxylic acid salts as the active ingredients. Individually, the preferred carboxylic acid salts of the present invention have antifungal activity against T. mentogrophytes and T. rubrum. However, when combined the resultant antifungal activity is synergistic. In this case, synergistic is defined to mean more than additive.

### EXAMPLE 4

### Demonstration of Synergistic Activity: Addition of Sodium Benzoate to Mixture of Zinc Propionate, Calcium Propionate and Sodium Formate

Using CLSI Reference Method 38-A2, activity against T. rubrum was examined using separately a mixture of zinc propionate (F9), calcium propionate (F10) and sodium formate (F12), sodium benzoate (F11) and a combination of zinc propionate, calcium propionate, sodium formate and sodium benzoate (N=negative growth; P=positive growth):

F9 + F10 + F12 VS F11

| | | | | | | | | |
|---|---|---|---|---|---|---|---|---|
| 0.25X | N | N | N | N | N | N | N | N |
| 0.125X | N | N | N | N | N | N | N | N |
| 0.06X | N | N | N | N | N | N | N | N |
| 0.03X | N | N | N | N | N | N | N | N |
| 0.015X | P | N | N | N | N | N | N | N |
| 0.007X | P | N | N | N | N | N | N | N |
| 0.004X | P | N | N | N | N | N | N | N |
| | | P 0.004X | P 0.007X | P 0.015X | N 0.03X | N 0.06X | N 0.125X | N 0.25X |

In this test, individually the combination of F9+F10+F12 showed negative growth at 0.03 times the initial concentration and F11 showed negative growth at 0.03 times the initial concentration. The combination of F9+F10+F12+F11 showed a negative growth at 0.004 times the concentration of F9+F10+F12 and 0.004 times the concentration of F11 thus demonstrating that the combination of the four carboxylic acid salts is synergistic.

### EXAMPLE 5

### Demonstration of Synergistic Activity: Addition of Zinc Propionate to a Mixture of Calcium Propionate, Sodium Benzoate and Sodium Formate

Using the same protocol as in Example 4 activity against T. rubrum was examined using separately a mixture of calcium propionate (F10), sodium benzoate (F11) and sodium formate (F12), zinc propionate (F9) and combined a mixture of calcium propionate, sodium benzoate, sodium formate and zinc propionate (N=negative growth; P=positive growth):

F10 + F11 + F12 VS F9

| | | | | | | | | |
|---|---|---|---|---|---|---|---|---|
| 0.25X | N | N | N | N | N | N | N | N |
| 0.125X | N | N | N | N | N | N | N | N |
| 0.06X | P | P | N | N | N | N | N | N |
| 0.03X | P | P | N | N | N | N | N | N |
| 0.015X | P | P | N | N | N | N | N | N |
| 0.007X | P | P | P | P | N | N | N | N |
| 0.004X | P | P | P | P | N | N | N | N |
| | | P 0.004X | P 0.007X | P 0.015X | N 0.03X | N 0.06X | N 0.125X | N 0.25X |

In this test, individually the combination of F10+F11+F12 showed negative growth at 0.06 times the initial concentration and F9 showed negative growth at 0.03 times the initial concentration. The combination of F10+F11+F12+F9 showed a negative growth at 0.015 times the concentration of F10+F11+F12 and at 0.007 times the concentration of F9 thus demonstrating that the combination of the four carboxylic acid salts is synergistic.

### EXAMPLE

### Demonstration of Synergistic Activity: Addition of Sodium Formate to a Mixture of Zinc Propionate and Calcium Propionate

Using the same protocol as in Example 4 , activity against T. rubrum was examined using separately a mixture of zinc propionate (F9) and calcium propionate (F10), and sodium formate (F12) and a combined mixture of zinc propionate, calcium propionate and sodium formate (N=negative growth; P=positive growth):

F9 + F10 VS F12

| | | | | | | | | |
|---|---|---|---|---|---|---|---|---|
| 0.25X | N | N | N | N | N | N | N | N |
| 0.125X | N | N | N | N | N | N | N | N |
| 0.06X | N | N | N | N | N | N | N | N |
| 0.03X | P | N | N | N | N | N | N | N |
| 0.015X | P | N | N | N | N | N | N | N |
| 0.007X | P | P | P | P | P | N | N | N |
| 0.004X | P | P | P | P | P | N | N | N |
| | | P 0.004X | P 0.007X | P 0.015X | P 0.03X | N 0.06X | N 0.125X | N 0.25X |

In this test, individually the combination of F9+F10 showed negative growth at 0.06 times the initial concentration and F12 showed negative growth at 0.06 times the initial concentration. The combination of F9+F10+F12 showed a negative growth at 0.015 times the concentration of F9+F10 and 0.004 times the concentration of F12 thus demonstrating that the combination of the three carboxylic acid salts is synergistic.

### EXAMPLE 7

### Demonstration of Synergistic Activity: Addition of Sodium Acetate to the Liquid Formulation of Example 1 without Sodium Formate

Using the same protocol as in Example 4, activity against T. rubrum was examined using separately the liquid formulation of Example 1 without sodium formate (F2), and sodium acetate (F12) and the combined liquid formulation of Example 1 without sodium formate and sodium acetate (N=negative growth; P=positive growth):

F2 VS F14

| | | | | | | | | |
|---|---|---|---|---|---|---|---|---|
| 0.25X | N | N | N | N | N | N | N | N |
| 0.125X | N | N | N | N | N | N | N | N |
| 0.06X | N | N | N | N | N | N | N | N |
| 0.03X | N | N | N | N | N | N | N | N |
| 0.015X | N | N | N | N | N | N | N | N |
| 0.007X | P | P | P | P | N | N | N | N |
| 0.004X | P | P | P | P | N | N | N | N |
| | | P 0.004X | P 0.007X | P 0.015X | P 0.03X | P 0.06X | N 0.125X | N 0.25X |

In this test, individually the liquid formulation F2 showed negative growth at 0.015 times the initial concentration and F14 showed negative growth at 0.125 times the initial concentration. The combination of F2+F14 showed a negative growth at 0.004 times the concentration of F2 and 0.03 times the concentration of F14 thus demonstrating that the combination of the liquid formulation of Example 1 without sodium formate and sodium acetate is synergistic.

### EXAMPLE 8

### Antifungal Activity Against Candida

Using the broth assay of Example 3 the liquid formulation of Example 1 was tested against Candida albacans, Candida parapsilosis and Candida krusei. Against C. albicans an MIC value of 0.63% of the liquid formulation of Example 1 was determined.

Against C. parapsilosis an MIC value of 0.63% of the liquid formulation of Example 1 was determined. Against C. krusei an MIC value of 0.31% of the liquid formulation of Example 1 was determined.

### EXAMPLE 9

### Penetrating Agent Action

To the liquid formulation of Example 1 was added isopropanol or ethanol so that it comprised 25, 33, 50, 66, 75 or 80% of the weight of the formulation. The new formulations were applied to a human nail. The liquid formulation dried on the nail leaving no powdery residue indicating that the carboxylic acid salts were rapidly absorbed by the human nail.

### EXAMPLE 10

### Treatment of Onychomycosis and Fungal infection

In two individuals, each with fungal infections of the nail on all nails of both feet, the nails were first debrided to remove damaged nail material only. Following debridement, the liquid formulation of Example 1 was applied to each nail and the surrounding skin twice daily (morning and evening). Within two weeks, both individuals perceived a marked reduction in the sensation of irritation at the nails. Treatment was continued for eight weeks at which time cultures taken from one individual tested negative for nail fungus. Following cessation of treatment, reoccurrence of the infection was not reported by either individual. No irritation or other discomfort owing to the application of a composition of the invention was reported by either subject.

In two individuals, each with a fungal infection of the nail on a big toe, the liquid formulation of Example 1 was applied twice daily (morning and evening) for eight weeks at which time there was no evidence of infection. Prior to treatment the nails were not debrided. Following cessation of treatment, reoccurrence of the infection was not reported by either individual. No irritation or other discomfort owing to the application of a composition of the invention was reported by either subject.

One individual reported suffering from a fungal infection of the skin on the top and underside of the foot and between the toes. The gel formulation of Example 2 was applied twice daily to this area. Although treatment was continued for eight weeks, the subject reported that the redness and irritation had cleared within two days

One individual with fungal infections on the under arms and the back sides of the elbows was treated with the liquid formulation of Example 1 twice daily (morning and evening) for five days. Following cessation of treatment, both infections had been completely eradicated; no reoccurrence was reported. No irritation or other discomfort owing to the application of a composition of the invention was reported.

While the treatment of infected nails in the above examples relied upon an eight-week application period, this should not be taken as the minimum time required for successful eradication of nail infections. An eight-week period was selected based upon the treatment periods generally indicated for fungal nail infections. Minimum effective treatment times using the present compositions may be determined in a formalized clinical trial program designed for such an outcome.

A synergistic enhancement by the low molecular weight, hydrophilic carboxylic acid salts or derivatives was observed in the treatment of fungal infections of the skin. An aqueous solution of calcium and sodium propionate (constituents of the known antifungal agent Mycoban®, which is commonly used as a food preservative) led to an initial clearing of fungal infections of the skin, however, the infections reoccurred within two weeks following cessation of treatment. In contrast, the infections were eradicated when using a composition of the present invention containing propionic acid and benzoic acid salts in propylene glycol, with an even greater synergistic enhancement observed through the addition of a third organic acid.

### EXAMPLE 11

### Treatment of Herpes Simplex

An individual with medically confirmed genital Herpes treated himself with the formulation described in example 1. The individual applied the formulation on two consecutive days to the effected area of the penis. On the day following the first application, the cold sore had mostly disappeared. Forty-eight hours following the first application, the herpes cold sore had completely disappeared.

As many changes can be made to the provided examples without departing from the scope of the invention, it is intended that all material herein be interpreted as illustrative of the invention and not in a limiting sense.

1. A non-toxic, topical antifungal composition comprising a combination of at least three or more low molecular weight, hydrophilic organic acid salts and/or derivatives exhibiting a synergistic enhancement, dissolved in a non-aqueous carrier, wherein
the low molecular weight organic acid salts are selected from the group consisting of saturated and unsaturated, acyclic, branched and unbranched aliphatic carboxylic acids wherein the longest carbon chain has eight carbons, and aromatic carboxylic acids containing less than ten carbon atoms;
the non-aqueous carrier is comprised of one or more non-aqueous, non-volatile polyhydric solvents; and
wherein the combination of low molecular weight, hydrophilic organic acid salts or derivatives comprises between about 0.5% to about 50 by weight of the composition, and no single acid salt or derivative comprises more than about 75% by weight of the total organic acid salt content.

2. (canceled)

3. (canceled)

4. The composition of claim 1, wherein the organic acid salts are selected from the group of organic acids consisting of formic, acetic, propionic, butyric, valeric, caproic, enanthic, caprylic, glyceric, tartaric, gluconic, benzoic, mandelic, acrylic, acetoacetic, pyruvic, adipic, aldaric, fumaric, glutaric, maleic, sorbic, malic, malonic, oxalic, succinic, tartronic acid salts.

5.-8. (canceled)

9. The composition of claim 81 wherein the composition comprises about 5% sodium benzoate, about 5% zinc propionate, about 5% calcium propionate and about 3% sodium formate.

10. (canceled)

11. The composition of claim 10 wherein the composition comprises about 5% zinc propionate, about 5% calcium propionate, about 5% sodium benzoate and about 3% sodium acetate.

12. The composition of claim 1 wherein the non-aqueous carrier comprises a non-aqueous, non-volatile polyhydric solvent selected from the group consisting of glycerine, diglycerol, ethylene glycol, propylene glycol, low molecular weight polyglycols, such as polyethylene glycol or polypropylene glycol with molecular weight less than 500 gmol⁻¹.

13. The composition of claim 12 wherein the non-aqueous carrier comprises propylene glycol.

14. The composition of claim 13 wherein the non-aqueous carrier additionally comprises glycerol.

15. The composition of claim 1 additionally comprising a penetrating agent selected from the group consisting of methanol, ethanol, n-propanol, isopropanol, n-butanol, sec-butanol, tert-butanol and mixtures thereof.

16. The composition of claim 15 wherein the penetrating agent is isopropanol or ethanol.

17.-19. (canceled)

20. A method for the treatment of fungal infection or onychomycosis on an external surface of a subject; the method comprising;
applying the composition of claim 1 to the external surface of the subject.

21. The use of method according to claim 20 wherein the treatment is for the treatment of onychomycosis.

22.-52. (canceled)

53. A method for treating a herpes cold sore, the method comprising: applying to the herpes cold sore a non-toxic topical composition comprising:
a combination of at least three or more low molecular weight, hydrophilic organic acid salts or derivatives, dissolved in a non-aqueous carrier, wherein
the low molecular weight organic acid salts or derivatives are selected from the group consisting of saturated and unsaturated, acyclic, branched and unbranched aliphatic carboxylic acids wherein the longest carbon chain has eight carbons, and aromatic carboxylic acids containing fewer than nine carbon atoms;
the non-aqueous carrier is comprised of one or more non-aqueous, non-volatile polyhydric solvents; and
wherein the combination of low molecular weight organic acid salts comprises between about 0.5% to about 50% by weight of the composition, and no single acid salt or derivative comprises more than about 75% by weight of the total organic acid salt content.

54. (canceled)

55. (canceled)

56. The method according to claim 53 wherein the organic acid salts are selected from the group of organic acids consisting of formic, acetic, propionic, butyric, valeric, caproic, enanthic, caprylic, glyceric, tartaric, gluconic, benzoic, mandelic, acrylic, acetoacetic, pyruvic, adipic, aldaric, fumaric, glutaric, maleic, sorbic, malic, malonic, oxalic, succinic, tartronic acid salts.

57.-63. (canceled)

64. The method according to claim 53 wherein the composition comprises about 5% zinc propionate, about 5% calcium propionate, about 5% sodium benzoate and about 3% sodium acetate.

65. The method according to claim 53 wherein the composition comprises about 5% zinc propionate, about 5% calcium propionate, about 5% sodium benzoate and about 3% sodium formate.

66. The method according claim 53 wherein the non-aqueous carrier comprises a non-aqueous, non-volatile polyhydric solvent selected from the group consisting of glycerine, diglycerol, ethylene glycol, propylene glycol and low molecular weight polyglycols, such as polyethylene glycol or polypropylene glycol with molecular weight less than 500 gmol⁻¹.

67. The method according to claim 66 wherein the non-aqueous carrier comprises propylene glycol.

68. The method according to claim 67 wherein the non-aqueous carrier additionally comprises glycerol.

69.-83. (canceled)

84. A method for treating a Candida infection the method comprising applying to the Candida infection a non-toxic topical composition comprising:
a combination of at least three or more low molecular weight, hydrophilic organic acid salts or derivatives, dissolved in a non-aqueous carrier, wherein
the low molecular weight organic acid salts or derivatives are selected from the group consisting of saturated and unsaturated, acyclic, branched and unbranched aliphatic carboxylic acids wherein the longest carbon chain has eight carbons, and aromatic carboxylic acids containing fewer than nine carbon atoms;
the non-aqueous carrier is comprised of one or more non-aqueous, non-volatile polyhydric solvents; and
wherein the combination of low molecular weight organic acid salts comprises between about 0.5% to about 50% by weight of the composition, and no single acid salt or derivative comprises more than about 75% by weight of the total organic acid salt content.

85. (canceled)

86. (canceled)

87. The method according to claim 8 wherein the organic acid salts are selected from the group of organic acids consisting of formic, acetic, propionic, butyric, valeric, caproic, enanthic, caprylic, glyceric, tartaric, gluconic, benzoic, mandelic, acrylic, acetoacetic, pyruvic, adipic, aldaric, fumaric, glutaric, maleic, sorbic, malic, malonic, oxalic, succinic, tartronic acid salts.

88.-91. (canceled)

92. The method according to claim 84 wherein the composition comprises about 5% zinc propionate, about 5% calcium propionate, about 5% sodium benzoate and about 3% sodium formate.

93. (canceled)

94. The method according to claim 84 wherein the composition comprises about 5% zinc propionate, about 5% calcium propionate, about 5% sodium benzoate and about 3% sodium acetate.

95. The method according to claim 84 wherein the non-aqueous carrier comprises a non-aqueous, non-volatile polyhydric solvent selected from the group consisting of glycerine, diglycerol, ethylene glycol, propylene glycol and low molecular weight polyglycols, such as polyethylene glycol or polypropylene glycol with molecular weight less than 500 gmol⁻¹.

96. The method according to claim 95 wherein the non-aqueous carrier comprises propylene glycol.

97. The method according to claim 96 wherein the non-aqueous carrier additionally comprises glycerol.
